(19) United States
(12) Patent Application Publication
(10) Pub. No.: US 2015/0238391 A1
(43) Pub. Date: Aug. 27, 2015

(54) BLEACHING AGENTS

(71) Applicant: Henkel AG & Co. KGaA, Duesseldorf (DE)

(72) Inventors: Juergen Schoepgens, Schwalmtal (DE); Frank Janssen, Koeln (DE); Bernd Anderheggen, Moenchengladbach (DE)

(21) Appl. No.: 14/711,976

(22) Filed: May 14, 2015

Related U.S. Application Data
(63) Continuation of application No. PCT/EP2013/071392, filed on Oct. 14, 2013.

(30) Foreign Application Priority Data
Nov. 14, 2012 (DE) 10 2012 220 807.0

(51) Int. Cl. A61K 8/02; A61K 8/92

(52) U.S. Cl. CPC A61K 8/022; A61K 8/92; A61Q 5/08

(57) ABSTRACT

Solid cosmetic agents, which contain a) at least one liquid fatty substance a), b) at least one powdery organic carrier material b), c) at least one cosmetic active agent c) that is different from a) and b), characterized in that the fatty substance a) and the powdery organic carrier material b) are in the form of a powdery aggregate A, the proportion by weight of the fatty substance a) relative to the total weight of this powdery aggregate A being higher than the proportion by weight of the fatty substance a) relative to the total weight of the solid cosmetic agent, are characterized by an above-average care effect in the bleaching of keratinic fibers.

### FIELD OF THE INVENTION

The present invention generally relates to a solid cosmetic agent, and more particularly relates to an agent for lightening or bleaching keratinic fibers, preferably human hair. The cosmetic agent is characterized by a content of fatty substances and carrier materials. Efficient lightening results with reduced hair damage and lower scalp irritation can be achieved in this way.

### BACKGROUND OF THE INVENTION

Changing the shape and color of the hair is an important area of modern cosmetics. It allows the appearance of the hair to be adapted both to the latest fashion trends and to the personal preferences of the individual. The basic principles of bleaching methods are known to the person skilled in the art and are described in relevant monographs.

For the bleaching of human hair, solid or paste-like preparations with solid oxidizing agents are conventionally mixed immediately before application with a dilute hydrogen peroxide solution. This mixture is then applied to the hair and rinsed out again after a certain contact time. The contact time on the hair in order to achieve complete lightening is between around 30 and 60 minutes. It is obvious that there is a need among users of these bleaching agents to reduce this contact time. To achieve an adequate bleaching effect, such agents are conventionally made highly alkaline; the pH is between 9 and 11.5. Such high pH values are necessary to ensure that the external cuticle opens and to allow the active species (hydrogen peroxide) to penetrate into the hair.

As well as having a bleaching effect, however, the use of oxidizing agents and the alkaline pH cause damage to the hair structure and to the surface of the hair. The hair becomes brittle, its elasticity decreases and it becomes less easy to comb. The splaying of the external cuticle leads moreover to an unpleasant surface sensitivity of the hair, making it more difficult to comb when wet and dry. Furthermore, alkalizing agents and oxidizing agents can lead to irritation and inflammation of the scalp, especially in sensitive users. For the consumer there is therefore a greater need to use additional conditioning or caring post-treatment agents.

The care agents which are used are mostly not sufficiently stable under the aggressive conditions (strong oxidizing agents, high pH) of the lightening preparation and of the cream preparations which are customary in the prior art (mostly with pH>11). Therefore the bleaching step and the conditioning agents usually have to follow on from one another. In the interests of convenience of use, however, a single-stage bleaching process, which already contains corresponding care agents, is particularly advantageous. There is therefore a need for bleaching agents which through the addition of stable care components to the application preparation lead to a decrease in hair damage and to reduced skin irritation.

German patent application DE 10 2009 054 763 A1 teaches the use of non-ionic surfactants and ester oils to improve the care effect of bleaching agents. German patent application DE 42 07 475 A1 describes bleaching agents which in addition to a starch powder additionally contain a fatty substance to reduce dust formation during production and use. The technical solutions described in these documents still leave room for improvement.

### BRIEF SUMMARY OF THE INVENTION

A solid cosmetic agent, containing at least one fatty substance a) having a melting point below 20° C.; at least one powdery organic carrier material b); and at least one cosmetic active agent c) that is different from a) and b), characterized in that the liquid fatty substance a) and the powdery organic carrier material b) are in the form of a powdery aggregate A, the proportion by weight of the liquid fatty substance a) relative to the total weight of this powdery aggregate A being higher than the proportion by weight of the liquid fatty substance a) relative to the total weight of the solid cosmetic agent.

A method for producing a solid cosmetic agent containing at least one fatty substance a) having a melting point below 20° C.; at least one powdery organic carrier material b); and at least one cosmetic active agent c) that is different from a) and b), characterized in that a powdery aggregate A, comprising at least one liquid fatty substance a) and at least one powdery organic carrier material b) is provided; and the aggregate A from step i) is mixed with at least one cosmetic active agent c).

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The object of the present invention is therefore to reduce the aforementioned disadvantages of oxidative lightening and/or bleaching agents for human hair. The lightening agents protect the hair, thereby leading to reduced damage to the hair. In particular, the agents impart greater elasticity and softness to the hair, along with improved shine. Furthermore, scalp irritation should be reduced. At the same time, however, the reduction in damage to the hair during lightening should not be achieved at the expense of a reduced lightening capacity of the agents.

It has been found that by using carrier-bound fatty substances in bleaching agents, the hair-damaging effect of such agents can be considerably reduced.

Furthermore, other desirable features and characteristics of the present invention will become apparent from the subsequent detailed description of the invention and the appended claims, taken in conjunction with the accompanying drawings and this background of the invention.

The invention firstly provides a solid cosmetic agent, containing

a) at least one fatty substance a) having a melting point below 20° C.
b) at least one powdery organic carrier material b)
c) at least one cosmetic active agent c) that is different from a) and b),
characterized in that the fatty substance a) and the powdery organic carrier material b) are in the form of a powdery aggregate A, the proportion by weight of the fatty substance a) relative to the total weight of this powdery aggregate A being higher than the proportion by weight of the fatty substance a) relative to the total weight of the solid cosmetic agent.

The solid cosmetic agent is preferably in powder form. It has proved advantageous for the caring effect of this agent to apply as large a proportion as possible of the fatty substance a) present in the agent to the powdery organic carrier material b). Preferred solid cosmetic agents are therefore characterized in that at least 40 wt. %, by preference at least 60 wt. %, preferably at least 80 wt. %, particularly preferably at least 90 wt. % and in particular at least 95 wt. % of the fatty substance a) present in the agent is in the form of the powdery aggregate A. It is particularly preferable for at least 98 wt. % and by preference all of the fatty substance a) present in the agent to be in the form of the powdery aggregate A.

As a first essential constituent the agents according to the invention contain at least one fatty substance a) having a melting point below 20° C. Preferred fatty substances a) are selected from the group of oils, in particular from the group of silicone oils. The proportion by weight of the fatty substance a) relative to the total weight of the solid cosmetic agent is by preference 0.1 to 5.0 wt. %, preferably 0.1 to 2.0 wt. % and in particular 0.1 to 1.0 wt. %.

Triglycerides and mixtures of triglycerides can be used as natural oils. Preferred natural oils are coconut oil, (sweet) almond oil, walnut oil, peach kernel oil, apricot kernel oil, avocado oil, tea tree oil, soybean oil, sesame oil, sunflower oil, tsubaki oil, evening primrose oil, rice bran oil, palm kernel oil, mango kernel oil, lady's smock oil, thistle oil, macadamia nut oil, grape seed oil, amaranth seed oil, argan oil, bamboo oil, olive oil, wheatgerm oil, pumpkin seed oil, mallow oil, hazelnut oil, safflower oil, canola oil, sasanqua oil, jojoba oil, rambutan oil, cocoa butter and shea butter. Suitable mineral oils are in particular mineral oils, paraffin and isoparaffin oils and synthetic hydrocarbons.

A particularly advantageous care effect is achieved through the use of silicone oils as the fatty substance a). In a preferred embodiment the fatty substance a) is therefore selected from the group of silicones, preferably from the group of dialkyl and alkylaryl siloxanes, the dimethicones, which also include cyclomethicones, dimethiconols, dimethicone copolyols and amino-functional silicones.

The preferred dimethicones according to the invention can be both linear and branched and also cyclic or cyclic and branched. Linear dimethicones can be represented by the following structural formula (Si1):

(SiR¹₃)—O—(SiR²₂—O—)ₓ—(SiR¹₃) (Si1)

The residues R¹ and R² independently of one another each denote a methyl residue, a C2 to C30 linear, saturated or unsaturated hydrocarbon residue, a phenyl residue and/or an aryl residue. The residues R¹ and R² preferably independently of one another denote a methyl residue or a phenyl residue. The number x denotes a value from 10 to 50,000. The molecular weights of the dimethicones are between 1000 D and 10,000,000 D. Silicones with the INCI name Phenyl Trimethicone are preferred in particular.

Particularly preferred hair treatment agents according to the invention are characterized in that they contain at least one silicone of formula (Si1.1)

(CH₃)₃Si—[O—Si(CH₃)₂]ₓ—O—Si(CH₃)₃ (Si1.1),

in which x denotes a number from 0 to 100, preferably from 0 to 50, more preferably from 0 to 20 and in particular 0 to 10.

Branched dimethicones can be represented by the structural formula (Si1.2):

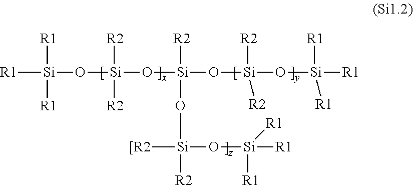

The residues R¹ and R² independently of one another each denote a methyl residue, a C2 to C30 linear, saturated or unsaturated hydrocarbon residue, a phenyl residue and/or an aryl residue. The numbers x, y and z are integers and each run independently of one another from 0 to 50,000. The molecular weights of the dimethicones are between 1000 D and 10,000,000 D.

The cyclic dimethicones referred to under INCI as cyclomethicones can also be used according to the invention. Cosmetic agents according to the invention are preferred here which contain at least one silicone of formula (Si-2)

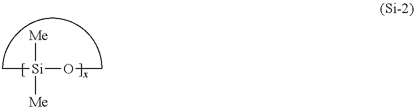

in which x denotes a number from 3 to 200, preferably from 3 to 10, more preferably from 3 to 7 and in particular 3, 4, 5 or 6.

Agents that are likewise preferred according to the invention are characterized in that they contain at least one silicone of formula (Si-3)

R₃Si—[O—SiR₂]ₓ—(CH₂)ₙ—[O—SiR₂]_{y}—O—SiR₃ (Si-3),

in which R denotes identical or different residues from the group —H, -phenyl, -benzyl, —CH₂—CH(CH₃)Ph, C₁-C₂₀ alkyl residues, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, x and y denote a number from 0 to 200, preferably from 0 to 10, more preferably from 0 to 7 and in particular 0, 1, 2, 3, 4, 5 or 6, and n denotes a number from 0 to 10, preferably from 1 to 8 and in particular 2, 3, 4, 5, 6.

A further group of preferred hair-caring silicones are the dimethiconols (Si4). Linear dimethiconols can be represented by the following structural formula (Si8-I):

(SiOHR¹₂)—O—(SiR²₂—O—)ₓ—(SiOHR¹₂) (Si4-1)

Branched dimethiconols can be represented by the structural formula (Si8-II):

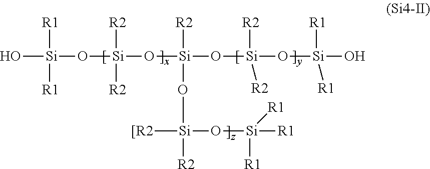

The residues R¹ and R² independently of one another denote hydrogen, a methyl residue, a C2 to C30 linear, saturated or unsaturated hydrocarbon residue, a phenyl residue and/or an aryl residue. The numbers x, y and z are integers and each run independently of one another from 0 to 50,000. The molecular weights of the dimethiconols are between 1000 D and 10,000,000 D.

Particularly preferred agents according to the invention contain one or more amino-functionalized silicones.

Particularly preferred agents according to the invention are characterized in that they contain at least one amino-functionalized silicone of formula (Si5-a)

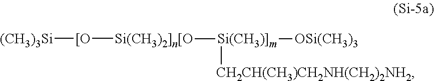

in which m and n are numbers whose sum (m+n) is between 1 and 2000, preferably between 50 and 150, wherein n preferably assumes values from 0 to 1999 and in particular from 49 to 149 and m preferably assumes values from 1 to 2000, in particular from 1 to 10. Under the INCI declaration these silicones are known as trimethylsilylamodimethicones.

Agents according to the invention which contain at least one amino-functionalized silane of formula (Si-5b)

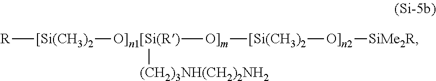

are also particularly preferred in which
R denotes —OH, an (optionally ethoxylated and/or propoxylated) (C₁ to C₂₀) alkoxy group or a —CH₃ group,
R′ denotes —OH, a (C₁ to C₂₀) alkoxy group or a —CH₃ group, and
m, n1 and n2 are numbers whose sum (m+n1+n2) is between 1 and 2000, preferably between 50 and 150, wherein the sum (n1+n2) preferably assumes values from 0 to 1999 and in particular from 49 to 149 and m preferably assumes values from 1 to 2000, in particular from 1 to 10.

Under the INCI declaration these silicones are known as amodimethicones or trimethylsiloxyamodimethicones, or functionalized amodimethicones, such as for example bis(C13-15 alkoxy) PG amodimethicone or trideceth-9 PG-amodimethicone.

Agents according to the invention which contain at least one amino-functionalized silane of formula (Si-5b)

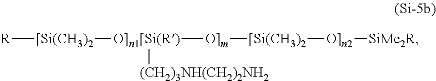

are preferred in particular in which
R denotes —OH or a —CH₃ group,
R′ denotes —OH or a —CH₃ group, and
m, n1 and n2 are numbers whose sum (m+n1+n2) is between 1 and 2000, preferably between 50 and 150, wherein the sum (n1+n2) preferably assumes values from 0 to 1999 and in particular from 49 to 149 and m preferably assumes values from 1 to 2000, in particular from 1 to 10.

Agents according to the invention which contain at least one amino-functionalized silane of formula (Si-5b)

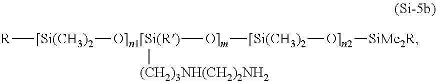

are moreover preferred in which
R denotes —OH or an —OCH₃ group,
R′ denotes —OH or an —OCH₃ group, and
m, n1 and n2 are numbers whose sum (m+n1+n2) is between 1 and 2000, preferably between 50 and 150, wherein the sum (n1+n2) preferably assumes values from 0 to 1999 and in particular from 49 to 149 and m preferably assumes values from 1 to 2000, in particular from 1 to 10.

In summary, preferred cosmetic agents contain at least one fatty substance a) from the group of silicones having the INCI names dimethicone, cyclomethicone, phenyl trimethicone, amodimethicone, trimethylsilylamodimethicone and trimethylsiloxyamodimethicone. The cosmetic agents particularly preferably contain as the fatty substance a) a mixture of an amino-functionalized silicone and a non-amino-functionalized silicone.

As a second essential constituent the agents according to the invention contain a powdery organic carrier material. The proportion by weight of the powdery organic carrier material b) relative to the total weight of the solid cosmetic agent is 0.2 to 10 wt. %, preferably 0.5 to 6.0 wt. % and in particular 1.0 to 4.0 wt. %.

Starch is suitable in particular as the organic carrier material b). Solid cosmetic agents characterized in that the powdery organic carrier material b) is selected from the group of starches are preferred according to the invention.

Starch is a reserve carbohydrate which is stored by many plants in the form of starch grains of conventionally 1 to 200 μm in size in various parts of the plant, for example in tubers or roots, cereal seeds, fruits and in the pith. A starch that can be used according to the invention can be obtained for example from potatoes, maize, rice, peas, acorns, chestnuts, barley, wheat, bananas, sago, millet, sorghum, oats, barley, rye, beans, yams, arrowroot or cassava.

Starch belongs to the homoglycan family and is a polycondensation product of D-glucose. Starch consists of three structurally different polymers of d-glucopyranose, namely amylose, amylopectin and an intermediate fraction. Vascular plants contain 0 to 45 wt. % of amylose relative to dry matter. In structural terms the intermediate fraction, which is also known as abnormal amylopectin, is between amylose and amylopectin.

In the context of this application the term “aggregate” refers to an aggregation of fatty substance a) and carrier material b) based on absorption and/or adsorption. The proportion by weight of the powdery aggregate A relative to the total weight of the solid cosmetic agent is preferably 0.5 to 10 wt. %, in particular 1.0 to 8.0 wt. %.

For the production and use of cosmetic agents according to the invention and for the desired care effect it has proved advantageous to use the fatty substance a) and the organic carrier material b) in specific weight ratios. Preferred cosmetic agents according to the invention are therefore characterized in that the weight ratio of liquid fatty substance a) to powdery organic carrier material b) in the powdery aggregate A is 2:1 to 1:20, preferably 1:1 to 1:12 and in particular 1:2 to 1:8.

As a further essential constituent the cosmetic agents according to the invention contain at least one cosmetic active agent c) that is different from the fatty substance a) and the carrier material b). As stated in the introductory part, the agents according to the invention are suitable in particular for gentle hair bleaching. Preferred cosmetic active agents c) are therefore selected from the group of constituents that are typical for hair bleaching agents, in particular from the group of bleaching agents, alkaline carriers, complexing agents and surfactants.

Preferred agents according to the invention contain as the cosmetic active agent c) at least one persulfate salt, preferably selected from the group of potassium peroxodisulfate, sodium peroxodisulfate and ammonium peroxodisulfate, as a bleaching strength intensifier. In the work on the present invention it has furthermore proved particularly preferable for the bleaching powder to contain at least two different peroxodisulfates. Preferred peroxodisulfate salts are combinations of ammonium peroxodisulfate and potassium peroxodisulfate and/or sodium peroxodisulfate. Preferred cosmetic agents contain 10 to 60 wt. %, preferably 15 to 55 wt. % and in particular 20 to 50 wt. % of an oxidizing agent, preferably a persulfate.

To further increase the lightening capacity at least one optionally hydrated SiO₂ compound can be added to the agents according to the invention. The lightening capacity can be increased significantly in this way. Preferred optionally hydrated SiO₂ compounds are silicic acids, oligomers and polymers thereof and salts thereof, in particular sodium salts, and water glasses formed from a silicate of the formula (SiO₂)ₙ(Na₂O)ₘ(K₂O)ₚ, where n denotes a positive rational number and m and p independently of one another denote a positive rational number or 0, with the provisos that at least one of the parameters m or p is different from 0 and that the ratio of n to the sum of m and p is between 1:4 and 4:1. Metasilicates, which are characterized according to the formula above by a ratio of n to the sum of m and p of ≦1 and which can be regarded as chain-like polymeric structures of the anion [SiO₃]²⁻, can also preferably be used. Sodium metasilicate of the formula [NaSiO₃]ₓ is particularly preferred. However, particularly preferred bleaching powders additionally contain at least silicon dioxide (silicic acid; silica gel). Preferred cosmetic agents are characterized in that they contain, relative to their total weight, 10 to 60 wt. %, preferably 15 to 55 wt. % and in particular 20 to 50 wt. % of a silicate.

Moreover, preferred cosmetic agents additionally contain a solid alkalizing agent. Alkalizing agents that can be used are selected from inorganic salts, in particular alkali and alkaline-earth metals, and solid, organic alkalizing agents, in particular basic amino acids. The preferred inorganic alkalizing agents according to the invention are preferably selected from the group of sodium hydroxide, potassium hydroxide, calcium hydroxide, barium hydroxide, sodium phosphate, potassium phosphate, sodium silicate, potassium silicate, sodium carbonate and potassium carbonate and mixtures thereof. The cosmetic agent contains the solid alkalizing agent preferably in amounts from 0.2 to 25 wt. %, in particular 0.5 to 10 wt. %, relative in each case to the total weight of the cosmetic agent.

It has proved advantageous according to the invention to formulate the cosmetic agent to be anhydrous. Anhydrous within the meaning of the present invention means a water content relative to the cosmetic agent of less than 5 wt. %, in particular less than 2 wt. %. Cosmetic agents containing less than 0.1 wt. % of water can be most particularly preferred according to the invention.

The composition of a number of preferred solid cosmetic agents according to the invention can be taken from the tables below (amounts in wt. % relative to the total weight of the solid cosmetic agent).

These agents are characterized in that
the liquid fatty substance a) and the powdery organic carrier material b) are in the form of a powdery aggregate A, the proportion by weight of the liquid fatty substance a) relative to the total weight of this powdery aggregate A being higher than the proportion by weight of the liquid fatty substance a) relative to the total weight of the solid cosmetic agent,
at least 40 wt. %, by preference at least 60 wt. %, preferably at least 80 wt. %, particularly preferably at least 90 wt. % and in particular at least 95 wt. % of the liquid fatty substance a) present in the agent is in the form of the powdery aggregate A.

| | Formula | Formula | Formula | Formula | Formula |
|---|---|---|---|---|---|
| | 1 | 2 | 3 | 4 | 5 |
| Liquid fatty | 0.1 to 5.0 | 0.1 to 2.0 | 0.1 to 2.0 | 0.1 to 1.0 | 0.1 to 1.0 |
| substance a) | | | | | |
| Powdery organic | 0.2 to 10 | 0.2 to 10 | 0.5 to 6.0 | 0.5 to 6.0 | 1.0 to 4.0 |
| carrier material b) | | | | | |
| Persulfate | 10 to 60 | 15 to 55 | 15 to 55 | 20 to 50 | 20 to 50 |
| | Formula | Formula | Formula | Formula | Formula |
| | 6 | 7 | 8 | 9 | 10 |
| Silicone | 0.1 to 5.0 | 0.1 to 2.0 | 0.1 to 2.0 | 0.1 to 1.0 | 0.1 to 1.0 |
| Powdery organic | 0.2 to 10 | 0.2 to 10 | 0.5 to 6.0 | 0.5 to 6.0 | 1.0 to 4.0 |
| carrier material b) | | | | | |
| Persulfate | 10 to 60 | 15 to 55 | 15 to 55 | 20 to 50 | 20 to 50 |
| | Formula | Formula | Formula | Formula | Formula |
| | 11 | 12 | 13 | 14 | 15 |
| Liquid fatty | 0.1 to 5.0 | 0.1 to 2.0 | 0.1 to 2.0 | 0.1 to 1.0 | 0.1 to 1.0 |
| substance a) | | | | | |
| Starch | 0.2 to 10 | 0.2 to 10 | 0.5 to 6.0 | 0.5 to 6.0 | 1.0 to 4.0 |
| Persulfate | 10 to 60 | 15 to 55 | 15 to 55 | 20 to 50 | 20 to 50 |
| | Formula | Formula | Formula | Formula | Formula |
| | 16 | 17 | 18 | 19 | 20 |
| Silicone | 0.1 to 5.0 | 0.1 to 2.0 | 0.1 to 2.0 | 0.1 to 1.0 | 0.1 to 1.0 |
| Starch | 0.2 to 10 | 0.2 to 10 | 0.5 to 6.0 | 0.5 to 6.0 | 1.0 to 4.0 |
| Persulfate | 10 to 60 | 15 to 55 | 15 to 55 | 20 to 50 | 20 to 50 |
| | Formula | Formula | Formula | Formula | Formula |
| | 21 | 22 | 23 | 24 | 25 |
| Silicone | 0.1 to 5.0 | 0.1 to 2.0 | 0.1 to 2.0 | 0.1 to 1.0 | 0.1 to 1.0 |
| Starch | 0.2 to 10 | 0.2 to 10 | 0.5 to 6.0 | 0.5 to 6.0 | 1.0 to 4.0 |
| Persulfate | 10 to 60 | 15 to 55 | 15 to 55 | 20 to 50 | 20 to 50 |
| Silicate | 10 to 60 | 15 to 55 | 15 to 55 | 20 to 50 | 20 to 50 |

The solid cosmetic agent according to the invention is suitable in particular for lightening human hair. Particularly effective lightening agents are characterized in that, in addition to the solid cosmetic agents described above, they additionally comprise at least one oxidizing preparation. The present application therefore also provides a cosmetic encompassing

A) a solid cosmetic agent, containing
a) at least one fatty substance a) having a melting point below 20° C.
b) at least one powdery organic carrier material b)
c) at least one cosmetic active agent c) that is different from a) and b), characterized in that the liquid fatty substance a) and the powdery organic carrier material b) are in the form of a powdery aggregate A, the proportion by weight of the liquid fatty substance a) relative to the total weight of this powdery aggregate A being higher than the proportion by weight of the liquid fatty substance a) relative to the total weight of the solid cosmetic agent A);
   B) an oxidizing preparation, containing
a) a liquid cosmetic carrier
b) hydrogen peroxide
c) at least one surfactant, preferably at least one non-ionic surfactant.

A liquid cosmetic carrier is aqueous or alcoholic or aqueous-alcoholic. Aqueous-alcoholic carriers are understood to be water-containing compositions containing 3 to 70 wt. % of a C₁ to C₄ alcohol, relative to the total weight of the oxidizing preparation, in particular ethanol or isopropanol. The agents according to the invention can additionally contain further organic solvents, such as for example 4-methoxybutanol, ethyl diglycol, 1,2-propylene glycol, n-propanol, n-butanol, n-butylene glycol, glycerol, diethylene glycol monoethyl ether, and diethylene glycol mono-n-butyl ether. All water-soluble organic solvents are preferred here. An aqueous carrier preferably contains at least 30 wt. %, in particular at least 50 wt. %, of water, relative to the total weight of the oxidizing preparation. For the purposes of hair lightening and/or bleaching, such carriers are for example creams, emulsions, gels or surfactant-containing foaming solutions, such as for example shampoos, foam aerosols or other preparations which are suitable for use on the hair. Preferred carriers are emulsions and gels, with emulsions being particularly preferred.

The oxidizing preparation contains hydrogen peroxide as an oxidizing agent. Hydrogen peroxide can be present as an aqueous solution or in the form of one of its addition products with organic or inorganic compounds. Preferred addition products are the addition products of hydrogen peroxide with urea, melamine and sodium borate. However, hydrogen peroxide in the form of an aqueous solution is preferably used as the oxidizing agent. The amount of hydrogen peroxide in the oxidizing agent preparation is preferably 3 to 25 wt. %, preferably 6 to 20 wt. % (calculated as 100% H₂O₂), relative in each case to the total weight of the oxidizing preparation.

As a further essential ingredient the oxidizing preparation contains a surfactant, preferably a non-ionic surfactant, in particular at least one ethoxylated fatty alcohol having 10 to 30 ethylene oxide units. According to the invention this is understood to be an addition product of ethylene oxide with a fatty alcohol. Fatty alcohols are saturated and unsaturated alcohols having 12 to 24 C atoms, which can be linear or branched. The molar amount of ethylene oxide that was used per mol of fatty alcohol indicates the degree of ethoxylation. In particular, ethylene oxide addition products with octyl alcohol (capryl alcohol), nonyl alcohol (pelargonyl alcohol), undecyl alcohol, undec-10-en-1-ol, dodecyl alcohol (lauryl alcohol), 2,6,8-trimethyl-4-nonanol (isolauryl alcohol), tridecyl alcohol, tetradecyl alcohol (myristyl alcohol), pentadecyl alcohol, hexadecyl alcohol (cetyl/palmityl alcohol), heptadecyl alcohol, octadecyl alcohol (stearyl alcohol), isostearyl alcohol, (9Z)-octadec-9-en-1-ol (oleyl alcohol), (9E)-octadec-9-en-1-ol (elaidyl alcohol), (9Z,12Z)-octadeca-9,12-dien-1-ol (linoleyl alcohol), (9Z,12Z,15Z)-octadeca-9,12,15-trien-1-ol (linolenyl alcohol), nonadecan-1-ol (nonadecyl alcohol), eicosan-1-ol (eicosyl alcohol/arachyl alcohol), (9Z)-eicos-9-en-1-ol (gadoleyl alcohol), (5Z,8Z,11Z,14Z)-eicosa-5,8,11,14-tetraen-1-ol (arachidonyl alcohol), heneicosyl alcohol, docosyl alcohol (behenyl alcohol), (13Z)-docos-13-en-1-ol (erucyl alcohol) or (13E)-docosen-1-ol (brassidyl alcohol) are suitable as the non-ionic surfactant. It is likewise possible according to the invention to use mixtures of fatty alcohols obtained by selective mixing or obtained per se by extraction methods. Examples are coconut alcohol (mixture of C₈-C₁₈ fatty alcohols) or cetearyl alcohol (1:1 mixture of C₁₆ and C₁₈ fatty alcohols).

Degrees of ethoxylation from 15 to 25 are preferred in particular. Ethoxylated fatty alcohols which are preferred in particular are laureth-20, steareth-20, steareth-16, steareth-25, ceteth-20, ceteth-16, ceteth-15, ceteth-24, ceteth-25, ceteareth-20, ceteareth-23, ceteareth-15, ceteareth-25, oleth-20, oleth-15 and oleth-16, particularly preferably ceteareth-20.

The proportion of surfactant, preferably non-ionic surfactant, in particular ethoxylated fatty alcohol with 10 to 30 ethylene oxide units, relative to the total weight of the oxidizing preparation, is preferably 0.1 to 5 wt. % and preferably 0.5 to 3 wt. %.

To prevent a premature, undesired reaction of the ingredients with one another, the ingredients of the solid cosmetic agent A) and the oxidizing preparation B) are conveniently packaged separately from one another and brought into contact immediately before use.

The present invention therefore secondly provides a ready-to-use agent C) for lightening human hair, which is characterized in that it is produced by mixing the solid cosmetic agent A) and the oxidizing preparation B).

The amount of hydrogen peroxide as oxidizing agent in the ready-to-use agent C) is preferably 1 to 12 wt. %, preferably 2 to 10 wt. %, particularly preferably 3 to 8 wt. % (calculated as 100% H₂O₂), relative in each case to the total weight of the ready-to-use agent C).

The tables below disclose the composition of some preferred cosmetics, comprising

A) a solid cosmetic agent, containing
a) at least one fatty substance a) having a melting point below 20° C.
b) at least one powdery organic carrier material b)
c) at least one cosmetic active agent c) that is different from a) and b),
   B) an oxidizing preparation, containing
a) a liquid cosmetic carrier
b) hydrogen peroxide
c) at least one surfactant, preferably at least one non-ionic surfactant.

These cosmetics are characterized in that
the liquid fatty substance a) and the powdery organic carrier material b) are in the form of a powdery aggregate A, the proportion by weight of the liquid fatty substance a) relative to the total weight of this powdery aggregate A being higher than the proportion by weight of the liquid fatty substance a) relative to the total weight of the solid cosmetic agent A),
at least 40 wt. %, by preference at least 60 wt. %, preferably at least 80 wt. %, particularly preferably at least 90 wt. % and in particular at least 95 wt. % of the liquid fatty substance present in the agent A) is in the form of the powdery aggregate A.
   (Amounts in wt. % relative to the total weight of the solid cosmetic agent A) or the oxidizing preparation B)).

| Formula | Formula | Formula | Formula | Formula |
|---|---|---|---|---|
| 26 | 27 | 28 | 29 | 30 |

Solid cosmetic agent A)

| | | | | | |
|---|---|---|---|---|---|
| Liquid fatty | 0.1 to 5.0 | 0.1 to 2.0 | 0.1 to 2.0 | 0.1 to 1.0 | 0.1 to 1.0 |
| substance a) | | | | | |
| Powdery organic | 0.2 to 10 | 0.2 to 10 | 0.5 to 6.0 | 0.5 to 6.0 | 1.0 to 4.0 |
| carrier material b) | | | | | |
| Persulfate | 10 to 60 | 15 to 55 | 15 to 55 | 20 to 50 | 20 to 50 |

Oxidizing preparation B)

| Hydrogen peroxide | 3 to 25 | 3 to 25 | 6 to 20 | 6 to 20 | 6 to 20 |
|---|---|---|---|---|---|
| Surfactant | 0.1 to 5.0 | 0.1 to 5.0 | 0.1 to 5.0 | 0.5 to 3.0 | 0.5 to 3.0 |
| | Formula | Formula | Formula | Formula | Formula |
| | 31 | 32 | 33 | 34 | 35 |

Solid cosmetic agent A)

| | | | | | |
|---|---|---|---|---|---|
| Silicone | 0.1 to 5.0 | 0.1 to 2.0 | 0.1 to 2.0 | 0.1 to 1.0 | 0.1 to 1.0 |
| Powdery organic | 0.2 to 10 | 0.2 to 10 | 0.5 to 6.0 | 0.5 to 6.0 | 1.0 to 4.0 |
| carrier material b) | | | | | |
| Persulfate | 10 to 60 | 15 to 55 | 15 to 55 | 20 to 50 | 20 to 50 |

Oxidizing preparation B)

| Hydrogen peroxide | 3 to 25 | 3 to 25 | 6 to 20 | 6 to 20 | 6 to 20 |
|---|---|---|---|---|---|
| Surfactant | 0.1 to 5.0 | 0.1 to 5.0 | 0.1 to 5.0 | 0.5 to 3.0 | 0.5 to 3.0 |
| | Formula | Formula | Formula | Formula | Formula |
| | 36 | 37 | 38 | 39 | 40 |

Solid cosmetic agent A)

| | | | | | |
|---|---|---|---|---|---|
| Liquid fatty | 0.1 to 5.0 | 0.1 to 2.0 | 0.1 to 2.0 | 0.1 to 1.0 | 0.1 to 1.0 |
| substance a) | | | | | |
| Starch | 0.2 to 10 | 0.2 to 10 | 0.5 to 6.0 | 0.5 to 6.0 | 1.0 to 4.0 |
| Persulfate | 10 to 60 | 15 to 55 | 15 to 55 | 20 to 50 | 20 to 50 |

Oxidizing preparation B)

| Hydrogen peroxide | 3 to 25 | 3 to 25 | 6 to 20 | 6 to 20 | 6 to 20 |
|---|---|---|---|---|---|
| Surfactant | 0.1 to 5.0 | 0.1 to 5.0 | 0.1 to 5.0 | 0.5 to 3.0 | 0.5 to 3.0 |
| | Formula | Formula | Formula | Formula | Formula |
| | 41 | 42 | 43 | 44 | 45 |

Solid cosmetic agent A)

| | | | | | |
|---|---|---|---|---|---|
| Silicone | 0.1 to 5.0 | 0.1 to 2.0 | 0.1 to 2.0 | 0.1 to 1.0 | 0.1 to 1.0 |
| Starch | 0.2 to 10 | 0.2 to 10 | 0.5 to 6.0 | 0.5 to 6.0 | 1.0 to 4.0 |
| Persulfate | 10 to 60 | 15 to 55 | 15 to 55 | 20 to 50 | 20 to 50 |

Oxidizing preparation B)

| Hydrogen peroxide | 3 to 25 | 3 to 25 | 6 to 20 | 6 to 20 | 6 to 20 |
|---|---|---|---|---|---|
| Surfactant | 0.1 to 5.0 | 0.1 to 5.0 | 0.1 to 5.0 | 0.5 to 3.0 | 0.5 to 3.0 |
| | Formula | Formula | Formula | Formula | Formula |
| | 46 | 47 | 48 | 49 | 50 |

Solid cosmetic agent A)

| | | | | | |
|---|---|---|---|---|---|
| Silicone | 0.1 to 5.0 | 0.1 to 2.0 | 0.1 to 2.0 | 0.1 to 1.0 | 0.1 to 1.0 |
| Starch | 0.2 to 10 | 0.2 to 10 | 0.5 to 6.0 | 0.5 to 6.0 | 1.0 to 4.0 |
| Persulfate | 10 to 60 | 15 to 55 | 15 to 55 | 20 to 50 | 20 to 50 |
| Silicate | 10 to 60 | 15 to 55 | 15 to 55 | 20 to 50 | 20 to 50 |

Oxidizing preparation B)

| | | | | | |
|---|---|---|---|---|---|
| Hydrogen peroxide | 3 to 25 | 3 to 25 | 6 to 20 | 6 to 20 | 6 to 20 |
| Surfactant | 0.1 to 5.0 | 0.1 to 5.0 | 0.1 to 5.0 | 0.5 to 3.0 | 0.5 to 3.0 |

For the cosmetic effect of the agents according to the invention it has proved advantageous for the solid cosmetic agent A) and/or the oxidizing preparation B) to contain at least one stabilizer or complexing agent. Common chelating agents which are preferred in the context of the present invention are for example polycarboxylic acids, nitrogen-containing mono- or polycarboxylic acids, in particular ethylenediamine tetraacetic acid (EDTA), ethylenediamine disuccinic acid (EDDS) and nitrilotriacetic acid (NTA), geminal diphosphonic acids, in particular 1-hydroxyethane-1,1-diphosphonic acid (HEDP), aminophosphonic acids such as ethylenediamine tetra(methylene phosphonic acid) (EDTMP), diethylenetriamine penta(methylene phosphonic acid) (DTPMP), phosphonopolycarboxylic acids such as 2-phosphonobutane-1,2,4-tricarboxylic acid, and cyclodextrins, alkali stannates (sodium stannate), alkali pyrophosphates (tetrasodium pyrophosphate, disodium pyrophosphate), alkali phosphates (sodium phosphate), and phosphoric acid. According to the invention the solid cosmetic agent A) and/or the oxidizing preparation B) preferably contain 0.01 to 3 wt. %, preferably 0.05 to 1 wt. %, of complexing agent, relative in each case to the total weight thereof.

The agents according to the invention can moreover contain at least one substantive dye. In principle there are no limits to the choice of substantive dyes. Substantive dyes which can be used according to the invention are nitrophenylene diamines, nitroaminophenols, azo dyes, anthraquinones or indophenols. Substantive dyes are conventionally divided into anionic, cationic and non-ionic substantive dyes. However, it is particularly advantageous according to the invention for the substantive dyes to have adequate stability in respect of the harsh conditions of the bleaching process.

Preferred anionic substantive dyes are the compounds known under the names Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1, Acid Black 52 and tetrabromophenol blue. Preferred cationic substantive dyes are cationic triphenylmethane dyes, such as Basic Blue 7, Basic Blue 26, Basic Violet 2 and Basic Violet 14, aromatic systems which are substituted with a quaternary nitrogen group, such as Basic Yellow 57, Basic Red 76, Basic Blue 99, Basic Brown 16 and Basic Brown 17 and HC Blue 16, as well as Basic Yellow 87, Basic Orange 31 and Basic Red 51. Preferred non-ionic substantive dyes are HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, as well as 1,4-diamino-2-nitrobenzene, 2-amino-4-nitrophenol, 1,4-bis-(2-hydroxyethyl)amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]-benzoic acid, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid and 2-chloro-6-ethylamino-4-nitrophenol.

The agent preferably contains as the substantive dye at least one anionic substantive dye selected from bromophenol blue, tetrabromophenol blue and/or from the group of fluorescein dyes, Acid Red 92 (also D&C RED No. 28 or Phloxin B), Acid Red 98, Acid Red 94, Acid Red 87, or Acid Red 51, and/or at least one neutral, substantive dye selected from 4-[(2,3-dihydroxypropyl)amino]-3-nitro-1-trifluoromethylbenzene (HC Yellow 6), 1-chloro-2,4-bis[(2-hydroxyethyl)amino]-5-nitrobenzene (HC Yellow 10), 1-chloro-4-[(2-hydroxyethyl)amino]-3-nitrobenzene (HC Yellow 12) and 4-[(2-hydroxyethyl)amino]-3-nitro-1-trifluoromethylbenzene (HC Yellow 13).

Preferred dye combinations according to the invention are those containing at least the combination of tetrabromophenol blue and Acid Red 92; tetrabromophenol blue and Acid Red 98; tetrabromophenol blue and Acid Red 94; tetrabromophenol blue and Acid Red 87; or tetrabromophenol blue and Acid Red 51. Agents containing at least one dye combination of tetrabromophenol blue and Acid Red 92 are particularly preferred according to the invention.

It is particularly advantageous according to the invention for the total amount of substantive dyes in the ready-to-use agent to be 0.0001 to 2.5 wt. %, relative to the total weight of the ready-to-use agent C).

The agents according to the invention can moreover contain further active agents, auxiliary substances and additives, such as for example associative polymers with a fatty alkyl chain, cationic polymers, non-ionic polymers (vinylpyrrolidinone/vinyl acrylate copolymers, polyvinylpyrrolidinone, vinylpyrrolidinone/vinyl acetate copolymers, polyethylene glycols and polysiloxanes); zwitterionic and amphoteric polymers (acrylamidopropyl trimethylammonium chloride/acrylate copolymers and octylacrylamide/methyl methacrylate/tert-butyl aminoethyl methacrylate/2-hydroxypropyl methacrylate copolymers); anionic polymers (polyacrylic acids, crosslinked polyacrylic acids, vinyl acetate/crotonic acid copolymers, vinylpyrrolidinone/vinyl acrylate copolymers, vinyl acetate/butyl maleate/isobornyl acrylate copolymers, methyl vinyl ether/maleic acid anhydride copolymers and acrylic acid/ethyl acrylate/N-tert-butyl acrylamide terpolymers); thickening agents (agar-agar, guar gum, alginates, xanthan gum, gum arabic, karaya gum, carob seed meal, linseed gums, dextrans, cellulose derivatives, e.g. methyl cellulose, hydroxyalkyl cellulose and carboxymethyl cellulose, starch fractions and derivatives such as amylose, amylopectin and dextrins, clays such as for example bentonite or fully synthetic hydrocolloids such as for example polyvinyl alcohol); hair-conditioning compounds (phospholipids, such as soy lecithin, egg lecithin, cephalins and silicone oils); protein hydrolysates of plant or animal origin (elastin, collagen, keratin, milk protein, soy protein and wheat protein hydrolysates, condensation products thereof with fatty acids and quaternized protein hydrolysates); perfume oils, dimethyl isosorbide and cyclodextrins; active agents to improve the fiber structure (mono-, di- and oligosaccharides, glucose, maleic acid and lactic acid); defoaming agents such as silicones (dimethicone); dyes for coloring the agent; anti-dandruff active agents (piroctone olamine, zinc omadine and climbazole); light stabilizers (derivatized benzophenones, cinnamic acid derivatives and triazines); active agents (pantolactone, allantoin, pyrrolidinone carboxylic acids and salts thereof and also bisabolol); vitamins, provitamins and vitamin precursors, in particular those of groups A, B₃, B₅, B₆, C, E, F and H; plant extracts (from green tea, oak bark, stinging nettle, witch hazel, hops, chamomile, burdock, horsetail, whitethom, lime blossom, almond, aloe vera, pine, horse chestnut, sandalwood, juniper, coconut, mango, apricot, lemon, lychee, wheat, kiwi, melon, orange, grapefruit, sage, rosemary, birch, mallow, lady's smock, wild thyme, yarrow, thyme, melissa, moringa, restharrow, coltsfoot, marshmallow, meristem, ginseng and ginger); cholesterol; consistency modifiers (sugar esters, polyol esters or polyol alkyl ethers); fats and waxes (fatty alcohols, beeswax, montan wax and paraffins); swelling and penetrating substances (glycerol, propylene glycol monoethyl ether, carbonates, hydrogen carbonates, guanidines, ureas and primary, secondary and tertiary phosphates); opacifiers (latex, styrene/PVP and styrene/acrylamide copolymers); pearlescent agents (ethylene glycol mono- and distearate and PEG-3-distearate); propellants such as propane-butane mixtures, N₂O, dimethyl ether, CO₂ and air; antioxidants.

The person skilled in the art will select these further substances in accordance with the desired properties of the preparations. The preparations preferably contain the further active agents, auxiliary substances and additives in amounts from 0.01 to 25 wt. %, in particular 0.05 to 15 wt. %, relative to the total amount of the ready-to-use agent C).

The bleaching preparations preferably have a pH in the range from 7 to 12, preferably a pH in the range from 8.0 to 11.5. The pH values within the meaning of the present invention are pH values measured at a temperature of 22° C.

The present application secondly provides a method for producing a solid cosmetic agent, containing

a) at least one liquid fatty substance a)
b) at least one powdery organic carrier material b)
c) at least one cosmetic active agent c) that is different from a) and b),
characterized in that
i) a powdery aggregate A, comprising at least one liquid fatty substance a) and at least one powdery organic carrier material b) is provided; and
ii) the aggregate A from step i) is mixed with at least one cosmetic active agent c).

The present invention also provides the use of an agent according to the invention for lightening human hair.

All that has been stated in respect of the agents according to the invention applies with necessary alterations to further preferred embodiments of the method according to the invention and of the use according to the invention.

### EXAMPLES

Unless otherwise specified, the amounts given in the examples are percentages by weight. The following preparations were produced:

### 1 Oxidizing Agent Preparations (A) (in Wt. %)

| Raw material | A |
|---|---|
| Sodium benzoate | 0.04 |
| Dipicolinic acid | 0.10 |
| Disodium pyrophosphate | 0.10 |
| Potassium hydroxide, 50% | 0.24 |
| HEDP, 60% | 0.31 |
| Eumulgade F | 4.50 |
| Liquid paraffin | 17.0 |
| Hydrogen peroxide, 50% | 18.2 |
| Water | to 100 |

### 2 Bleaching Powder (B) (in Wt. %)

| Raw material | B1 | B2 |
|---|---|---|
| Potassium peroxodisulfate | 32.0 | 32.0 |
| Ammonium peroxodisulfate | 10.0 | 10.0 |
| (including 0.5% silica) | | |
| Silica, hydrophilic | 0.4 | 0.4 |
| Sodium metasilicate | 36.0 | 36.0 |
| Portil N | — | — |
| Sodium hexametaphosphate | 0.2 | 0.2 |
| Rohagit S hv | 1.0 | 1.0 |
| EDTA, Na₂ salt | 0.6 | 0.6 |
| Celquat L200 | 0.3 | 0.3 |
| Glycine | 0.6 | 0.6 |
| Liquid paraffin | 3.8 | 3.8 |
| DG XX-4820 powder | 4.0 | — |
| Dimethicone, dimethiconol | — | 1.5 |
| Magnesium carbonate | to 100 | to 100 |

Raw materials: Eumulgade F (INCI name: Cetearyl Alcohol, PEG-40 Castor Oil, Sodium Cetearyl Sulfate); Portil N (INCI name: Sodium Silicate; Cognis); Rohagit S hv (INCI name: Acrylates Copolymer; Evonik); Celquat L200 (INCI name: Polyquatemium-4; National Starch); DC XX-4820 powder (16 wt. % silicone on starch carrier).

The bleaching agent C1 according to the invention was obtained by mixing preparations A and B1 in the ratio 2:1. To produce the bleaching agent C2 preparations A and B2 were mixed together in the ratio 2:1.

To assess the wet combability, 12 hair strands in each case (Kerling 6/0) were first washed with a 3% aqueous surfactant solution and stored for 24 hours at 25° C. with 25% humidity and then treated for 45 minutes at 35° C. with 4 g of bleaching agent C1 or C2 in each case.

Following treatment with bleaching agents C1 and C2 the strands were rinsed with water under standard conditions (water temperature 32° C., 0.51/min). The strands were then each combed automatically ten times, while determining the energy expended. The averaged measuring results are set out in the table below.

| | Energy expended [mJ] |
|---|---|
| C1 | 704 |
| C2 | 798 |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A solid cosmetic agent, containing
a) at least one fatty substance having a melting point below 20° C.;
b) at least one powdery organic carrier material;
c) at least one cosmetic active agent that is different from the at least one fatty substance and the at least one powdery organic carrier,
wherein the at least one fatty substance and the powdery organic carrier material are in the form of a powdery aggregate A, the proportion by weight of the at least one fatty substance relative to the total weight of the powdery aggregate A being higher than the proportion by weight of the at least one fatty substance relative to the total weight of the solid cosmetic agent.

2. The solid cosmetic agent according to claim 1, wherein at least 40 wt. % of the at least one fatty substance in the solid cosmetic agent is in the form of the powdery aggregate A.

3. The solid cosmetic agent according to claim 1, wherein at least 60 wt. % of the at least one fatty substance in the solid cosmetic agent is in the form of the powdery aggregate A.

4. The solid cosmetic agent according to claim 1, wherein at least 90 wt. % of the at least one fatty substance in the solid cosmetic agent is in the form of the powdery aggregate A.

5. The solid cosmetic agent according to claim 1, wherein the fatty acid substance is a silicone.

6. The solid cosmetic agent according to claim 1, wherein the proportion by weight of the fatty substance relative to the total weight of the solid cosmetic agent is 0.1 to 5.0 wt. %.

7. The solid cosmetic agent according to claim 1, wherein the proportion by weight of the fatty substance relative to the total weight of the solid cosmetic agent is 0.1 to 2.0 wt. %.

8. The solid cosmetic agent according to claim 1, wherein the proportion by weight of the fatty substance relative to the total weight of the solid cosmetic agent is 0.1 to 1.0 wt. %.

9. The solid cosmetic agent according to claim 1, wherein the powdery organic carrier material is a starch.

10. The solid cosmetic agent according to claim 1, wherein the proportion by weight of the powdery organic carrier material relative to the total weight of the solid cosmetic agent is 0.2 to 10 wt. %.

11. The solid cosmetic agent according to claim 1, wherein the proportion by weight of the powdery organic carrier material relative to the total weight of the solid cosmetic agent is 1.0 to 4.0 wt. %.

12. The solid cosmetic agent according to claim 1, wherein the proportion by weight of the powdery aggregate A relative to the total weight of the solid cosmetic agent is 0.5 to 10 wt. %.

13. The solid cosmetic agent according to claim 1, wherein the proportion by weight of the powdery aggregate A relative to the total weight of the solid cosmetic agent is 1.0 to 8.0 wt. %.

14. The solid cosmetic agent according to claim 1, wherein relative to its total weight the solid cosmetic agent contains 10 to 60 wt. % of an oxidizing agent.

15. The solid cosmetic agent according to claim 14, wherein relative to its total weight the solid cosmetic agent contains 15 to 55 wt. % of an oxidizing agent.

16. The solid cosmetic agent according to claim 14, wherein relative to its total weight the solid cosmetic agent contains 20 to 50 wt. % of an oxidizing agent.

17. The solid cosmetic agent according to claim 14, wherein the oxidizing agent is a persulfate.

18. The solid cosmetic agent according to claim 1, wherein relative to its total weight the solid cosmetic agent contains 10 to 60 wt. % of a silicate.

19. The solid cosmetic agent according to claim 1, wherein relative to its total weight the solid cosmetic agent contains 20 to 50 wt. % of a silicate.

20. A method for producing a solid cosmetic agent containing
a) at least one fatty substance having a melting point below 20° C.;
b) at least one powdery organic carrier material;
c) at least one cosmetic active agent that is different from the at least one fatty substance and the at least one powdery organic carrier;
wherein
i) a powdery aggregate A, comprising at least one fatty substance and at least one powdery organic carrier material is provided; and
ii) the aggregate A is mixed with at least one cosmetic active agent.
